(19) United States
(12) Patent Application Publication
(10) Pub. No.: US 2013/0096521 A1
(43) Pub. Date: Apr. 18, 2013

(54) Polymeric Film Comprising An Odor Absorbing PVDC Blend

(71) Applicant: Solomon Bekele, Taylors, SC (US)

(72) Inventor: Solomon Bekele, Taylors, SC (US)

(73) Assignee: Cryovac, Inc, Duncan, SC (US)

(21) Appl. No.: 13/647,010

(22) Filed: Oct. 8, 2012

Related U.S. Application Data
(60) Provisional application No. 61/547,164, filed on Oct. 14, 2011.

(51) Int. Cl. A61F 5/441; C08K 3/22; B32B 38/04

(52) U.S. Cl. 604/333; 156/253; 524/433

(57) ABSTRACT

The presently disclosed subject matter is directed to a polymeric film that comprises at least one odor absorbent layer. Specifically, the odor absorbent layer can comprise polyvinylidene chloride polymer and magnesium oxide, magnesium salt, magnesium hydroxide, zeolite, or combinations thereof. The use of the disclosed film in ostomy pouches and other medical products is also described.

### FIELD OF THE INVENTION

The presently disclosed subject matter is directed to polymeric films that include at least one odor absorbing layer. The disclosed films can be suitable for medical use (such as for incorporation in an ostomy collection bag) to absorb unpleasant odors, thereby preventing or lessening transmission of such odors.

### BACKGROUND

It is well known in the art to provide ostomy pouches with filters to deodorize the escaping gases. Typically, such a filter takes the form of disc or pad composed of fibrous elements coated with finely-divided activated carbon particles. The disc is typically secured to the wall of a pouch over a vent opening. In an effort to prevent the filter from becoming clogged and rendered ineffective by liquid and/or solid body waste material, it has been common practice to secure the filter to the outside surface of the pouch over a vent opening. Alternatively, the filter can be protected by a hydrophobic and/or oleophobic porous membrane that extends over the filter.

Even when an ostomy filter is protected by a hydrophobic/oleophobic microporous membrane, liquid contact can still render the filter inoperative if the filter becomes saturated by water from an external source, such as when an ostomate wears an ostomy pouch while taking a shower. In such cases, water can enter the filter through the vent opening in the wall of the pouch. Efforts have been made to reduce these problems by constructing S-shaped vent openings, but it is recognized that such constructions do not completely solve the problem.

A main aspect of the presently disclosed subject matter therefore lies in providing a high-performance film that can be used to construct ostomy pouches. Specifically, the film comprises at least one odor-absorbing layer that functions to absorb odors emanating from the interior of the ostomy pouch. In addition, the disclosed film imparts a high oxygen barrier property to the pouch. The disclosed film also meets the fit-for-use requirements suitable for ostomy pouches (such as, for example, film quietness during movement). All of these features are achieved by the presently disclosed film which includes an odor-absorbing layer comprising a polyvinylidene chloride polymer blended with a magnesium oxide, magnesium salt, magnesium hydroxide, and/or a zeolite.

### SUMMARY

In some embodiments, the presently disclosed subject matter is directed to a polymeric film comprising an odor absorbent layer. The odor absorbent layer comprises polyvinylidene chloride polymer and (a) magnesium oxide, (b) magnesium salt, (c) magnesium hydroxide, (d) zeolite, or (e) combinations thereof.

In some embodiments, the presently disclosed subject matter is directed to an ostomy pouch comprising a polymeric film comprising an odor absorbent layer. The odor absorbent layer comprises polyvinylidene chloride polymer and (a) magnesium oxide, (b) magnesium salt, (c) magnesium hydroxide, (d) zeolite, or (e) combinations thereof.

In some embodiments, the presently disclosed subject matter is directed to a method of making an ostomy pouch. The method comprises forming a pouch comprising a first wall and a second wall joined together to define a closed compartment having an interior. The method also comprises providing an aperture formed in one of the first or second walls of the pouch and in communication with the pouch interior. The first and second walls comprise a polymeric film comprising an odor absorbent layer, wherein the odor absorbent layer comprises polyvinylidene chloride polymer and (a) magnesium oxide, (b) magnesium salt, (c) magnesium hydroxide, (d) zeolite, or (e) combinations thereof.

### BRIEF DESCRIPTION OF THE DRAWINGS

FIGS. 1-4 are schematic cross-sectional views of films in accordance with some embodiments of the presently disclosed subject matter.

FIG. 5 is a top plan view of one embodiment of an ostomy pouch that can be constructed from the disclosed film.

FIG. 6 is a line graph depicting the relative smell strength of chopped onion in a series of ostomy pouches.

FIG. 7 is a line graph depicting the onion odor permeation intensity of a series of ostomy pouches.

FIG. 8 is a bar graph illustrating the oxygen transmission rate of the presently disclosed films at 23° C. and 0% relative humidity.

FIG. 9 is a graph illustrating the dimethyl disulfide breakthrough time with Films 17-20.

### DETAILED DESCRIPTION

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long standing patent law convention, the terms “a”, “an”, and “the” refer to “one or more” when used in the subject application, including the claims. Thus, for example, reference to “a film” includes a plurality of such films, and so forth.

Unless indicated otherwise, all numbers expressing quantities of components, reaction conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount, as such variations are appropriated in the disclosed package and methods.

As used herein, the phrase “abuse layer” refers to an outer film layer and/or an inner film layer, so long as the film layer serves to resist abrasion, puncture, and other potential causes of reduction of package integrity, as well as potential causes of reduction of package appearance quality. Abuse layers can comprise any polymer, so long as the polymer contributes to achieving an integrity goal and/or an appearance goal; preferably, abuse layers comprise polymer having a modulus of at least 107 Pascals, at room temperature; more preferably, the abuse layer comprises at least one member selected from the group consisting of polyolefin (ethylene vinyl acetate, low density polyethylene, linear low density polyethylene, metallocene linear low density polyethylene, and the like), polyamide, ethylene/propylene copolymer; more preferably, nylon 6, nylon 6/6, amorphous nylon, and ethylene/propylene copolymer.

The term “antiblock agents” as used herein refers to particles that can be used to lower the coefficient of friction and/or adhesion of outer film surfaces, and includes various silicas (fumed, precipitated, gelled, etc.), natural silicates (talc, diatomaceous earth, etc.), magnesium silicate, carbonates, synthetic silicate, natural alumina, synthetic alumina, alumino silicate, synthetic particulates (uncrosslinked styrenic polymeric particles, crosslinked styrenic polymeric particles, high molecular and ultrahigh molecular weight siloxanes, uncrosslinked acrylic polymeric particles, crosslinked acrylic polymeric particles, polyethylene particles, styrenic, acrylic, siloxane, fluoropolymer, etc.), and the like. Other types of antiblock agents can include amidic waxes, such as the bis fatty amides.

As used herein, the terms “barrier” and “barrier layer” as applied to films and/or film layers, refer to the ability of a film or film layer to serve as a barrier to gases and/or odors. Examples of polymeric materials with low oxygen transmission rates useful in such a layer can include: ethylene/vinyl alcohol copolymer (EVOH), polyvinylidene dichloride (PVDC), vinylidene chloride copolymer such as vinylidene chloride/methyl acrylate copolymer, vinylidene chloride/vinyl chloride copolymer, polyamide, polyglycolic acid, polyester, polyacrylonitrile (available as Barex™ resin), or blends thereof. Oxygen barrier materials can further comprise high aspect ratio fillers that create a tortuous path for permeation (e.g., nanocomposites). Oxygen barrier properties can be further enhanced by the incorporation of an oxygen scavenger, such as an organic oxygen scavenger. In some embodiments, metal foil, metallized substrates (e.g., metallized polyethylene terephthalate (PET), metallized polyamide, and/or metallized polypropylene), and/or coatings comprising SiOx or AlOx compounds can be used to provide low oxygen transmission to a package. In some embodiments, a barrier layer can have a gas (e.g., oxygen) permeability of less than or equal to about 500 cc/m²/24 hrs/atm at 73° F., in some embodiments less than about 100 cc/m²/24 hrs/atm at 73° F., in some embodiments less than about 50 cc/m²/24 hrs/atm at 73° F., and in some embodiments less than about 25 cc/m²/24 hrs/atm at 73° F.

The term “bulk layer” as used herein refers to a layer used to increase the abuse-resistance, toughness, modulus, etc., of a film. In some embodiments, the bulk layer can comprise polyolefin (including but not limited to) at least one member selected from the group comprising ethylene/alpha-olefin copolymer, ethylene/alpha-olefin copolymer plastomer, low density polyethylene, and/or linear low density polyethylene and polyethylene vinyl acetate copolymers.

As used herein, the term “core”, and the phrase “core layer”, as applied to multilayer films, can refer to any inner (i.e., internal) film layer that has a primary function other than serving as an adhesive or compatibilizer for adhering two layers to one another. In some embodiments, the core layer or layers provide a film with a desired level of strength, i.e., modulus, and/or optics, and/or added abuse resistance, and/or specific impermeability.

The term “disposable” as used herein describes devices (such as ostomy pouches) that are generally not intended to be laundered or otherwise restored or reused (i.e., they are intended to be discarded after a single use).

As used herein, the term “film” can be used in a generic sense to include plastic web, regardless of whether it is film or sheet. In some embodiments, the term “film” can include a nonwoven fabric, paper tissue, and like materials.

The term “fluid” as used herein refers to liquids, gases, semi-solids, pastes, gels, and combinations thereof. In some embodiments, the term “fluid” can include any substance that is not a solid.

The term “magnesium hydroxide” as used herein refers to magnesium hydroxides of natural or synthetic origin. Natural magnesium hydroxides can be those obtained from seawater or from Mg(OH)₂-containing minerals, such as, brucite. Synthetic magnesium hydroxides can be those marketed, for example, by Martinswerk GmbH in Bergheim under the tradename Magnifin®. In some embodiments, the term magnesium hydroxide also covers magnesium hydroxycarbonates, for example, marketed by Microfine Minerals under the tradename Ultracarb®.

The term “magnesium oxide” as used herein includes MgO and can be purchased from a wide variety of vendors, such as Merck, VIGORTONE, Inc., Martin Marietta Chemicals, Aldrich, and the like.

The term “magnesium salt” as used herein includes the various known salts of magnesium, including (but not limited to) magnesium sulfate, magnesium chloride, magnesium bromide, magnesium iodide, magnesium acetate, magnesium nitrate, and the like, as well as various hydrates thereof.

As used herein, “oxygen transmission rate”, also referred to as “OTR” and “oxygen permeability”, is measured according to ASTM D 3985, a test known to those of skill in the film art. The content of ASTM D 3985 is hereby incorporated by reference in its entirety.

As used herein, the term “polymer” refers to the product of a polymerization reaction, and can be inclusive of homopolymers, copolymers, terpolymers, etc. In some embodiments, the layers of a film can consist essentially of a single polymer, or can have additional polymer together therewith, i.e., blended therewith.

The term “pouch” as used herein is not limiting and includes the wide variety of containers known in the art, including (but not limited to) bags, packets, packages, and the like. In some embodiments, the term “pouch” includes any protective or collective device having an opening adapted to be secured about a stoma for protecting the user and for collecting exudate. Such pouches are well known in the art; e.g., U.S. Pat. Nos. 3,827,435; 3,954,105; 4,205,678; 4,268,286; 4,983,171; and 5,074,851, the entire disclosures of which are hereby incorporated by reference herein.

As used herein, the term “seal” refers to any seal of a first region of an outer film surface to a second region of an outer film surface, including heat or any type of adhesive material, thermal or otherwise. In some embodiments, the seal can be formed by heating the regions to at least their respective seal initiation temperatures. The sealing can be performed by any one or more of a wide variety of means, including (but not limited to) using a heat seal technique (e.g., melt-bead sealing, thermal sealing, impulse sealing, dielectric sealing, radio frequency sealing, ultrasonic sealing, hot air, hot wire, infrared radiation, pressure sensitive adhesives, UV curing adhesive, and the like).

As used herein, the phrases “seal layer”, “sealing layer”, “heat seal layer”, and “sealant layer”, refer to an outer film layer, or layers, involved in the sealing of the film to itself, another film layer of the same or another film, and/or another article that is not a film. It should also be recognized that in general, up to the outer 3 mils of a film can be involved in the sealing of the film to itself or another layer. In general, a sealant layer sealed by heat-sealing layer comprises any thermoplastic polymer. In some embodiments, the heat-sealing layer can comprise, for example, thermoplastic polyolefin, thermoplastic polyamide, thermoplastic polyester, and thermoplastic polyvinyl chloride. In some embodiments, the heat-sealing layer can comprise thermoplastic polyolefin.

As used herein, the term “tie layer” refers to an internal film layer having the primary purpose of adhering two layers to one another. In some embodiments, tie layers can comprise any nonpolar polymer having a polar group grafted thereon, such that the polymer is capable of covalent bonding to polar polymers such as polyamide and ethylene/vinyl alcohol copolymer. In some embodiments, tie layers can comprise at least one member selected from the group including, but not limited to, modified polyolefin, modified ethylene/vinyl acetate copolymer, and/or homogeneous ethylene/alpha-olefin copolymer. In some embodiments, tie layers can comprise at least one member selected from the group consisting of anhydride modified grafted linear low density polyethylene, anhydride grafted low density polyethylene, homogeneous ethylene/alpha-olefin copolymer, and/or anhydride grafted ethylene/vinyl acetate copolymer.

The term “vinylidene chloride polymer” or “vinylidene chloride copolymer” or “PVDC” as used herein refers to vinylidene chloride copolymerized with at least one other monomer which includes (but is not limited to) vinyl chloride, C₁ to C₈ alkyl acrylates (such as methyl acrylate), C₁ to C₈ alkyl methacrylates and acrylonitrile.

All compositional percentages used herein are presented on a “by weight” basis, unless designated otherwise.

Although the majority of the above definitions are substantially as understood by those of skill in the art, one or more of the above definitions can be defined hereinabove in a manner differing from the meaning as ordinarily understood by those of skill in the art, due to the particular description herein of the presently disclosed subject matter.

### II. The Presently Disclosed Film

II.A. Generally

The presently disclosed subject matter is directed to a polymeric film suitable for use in a wide variety of applications, such as the formation of ostomy bags, incontinence bags, medical collection bags, and the like. Specifically, the packaging film incorporates at least one odor absorbent layer. As set forth in more detail herein below, the odor absorbent layer includes a polyvinylidene chloride polymer blended with a magnesium oxide, magnesium salt, magnesium hydroxide, and/or zeolite, resulting in a film that maintains excellent oxygen barrier properties, as well as provides enhanced odor absorbing capabilities. The odor absorbent layer can be a core layer, an inner layer, a sealant layer and/or an exterior layer, as set forth below.

The disclosed film can be monolayer or multilayer. To this end, the disclosed film can comprise 1 to 20 layers; in some embodiments, from 2 to 12 layers; in some embodiments, from 2 to 9 layers; and in some embodiments, from 3 to 8 layers. Thus, in some embodiments, the disclosed film can have 1, 2, 3, 4, 5, 6, 7, 8, 9, 10, 11, 12, 13, 14, 15, 16, 17, 18, 19, or 20 layers. One of ordinary skill in the art would also recognize that the disclosed film can comprise more than 20 layers, such as in embodiments wherein the film components comprise microlayering technology.

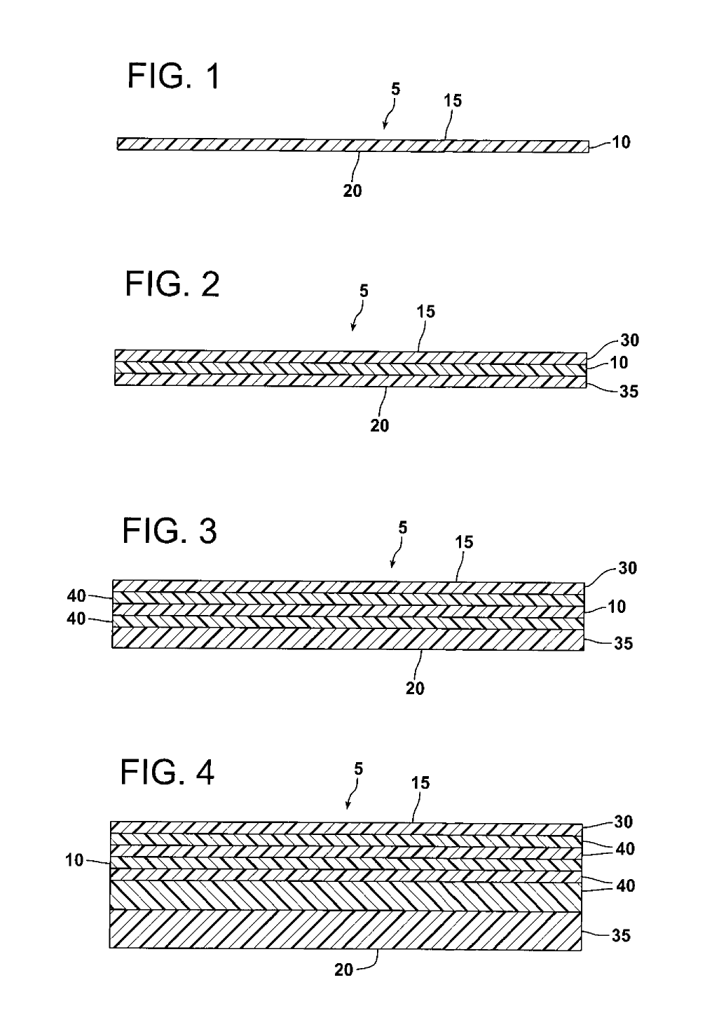

Accordingly, as illustrated in FIG. 1, in some embodiments, film 5 can be a monolayer film comprising core layer 10. Core layer 10 comprises interior surface 15 that is proximate to or contacting a packaged product. Core layer 10 also comprises exterior surface 20 positioned opposite to interior surface 15. Thus, in some embodiments, film 5 can by itself be considered an odor absorbent layer. As would be readily understood to those having ordinary skill in the art of film manufacture, a greater number of layers can be included, depending on the specific attributes that are desired in such a film to suit the intended end-use application.

For example, FIG. 2 illustrates a cross-sectional view of a film comprising 3 layers in accordance with the presently disclosed subject matter. In the embodiment depicted in FIG. 2, film 5 comprises interior surface 15 that is proximate to or contacting a packaged product and exterior surface 20 positioned opposite to interior surface 15. In addition, the film of FIG. 2 comprises sealant layer 30 and external layer 35. It is noted that in some embodiments FIG. 2 is not drawn to scale and layers 10, 30, and 35 can be of varying thicknesses compared to one another.

FIGS. 3 and 4 illustrate cross-sectional views of a multilayered film comprising 5 and 7 layers, respectively. In the embodiment depicted in FIGS. 3 and 4, film 5 comprises interior surface 15 that is proximate to or contacting a packaged product and exterior surface 20 positioned opposite to interior surface 15. In addition, the films of FIGS. 3 and 4 comprise core layer 10 and inner layers 40. An “inner layer” is a layer that has both of its surfaces directly adhered to other layers of the multilayer film. It is noted that in some embodiments FIGS. 3, and 4 are not drawn to scale and layers 10, 30, 35, and 40 can be of varying thicknesses compared to one another.

The disclosed film can have any total thickness as long as it provides the desired properties for the particular packaging operation in which it is to be used. Nevertheless, in some embodiments the disclosed film has a total thickness ranging from about 0.1 mil to about 15 mils; in some embodiments, from about 0.2 mil to about 10 mils; in some embodiments, from about 0.3 mils to about 5.0 mils; and in some embodiments, from about 1 mil to about 3 mils. Thus, in some embodiments, film 5 can have a thickness of about 0.1, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 mils.

The disclosed film can be cross-linked, depending upon the application in which the film is utilized. Cross-linking increases the structural strength of the film at elevated temperatures and/or increases the force at which the material can be stretched before tearing apart. Cross-linking can be achieved through the use of irradiation (i.e., bombarding the film with particulate or non-particulate radiation such as high-energy electrons from an accelerator or cobalt-60 gamma rays) to cross-link the materials of the film. In some embodiments, the irradiation dosage level can be in the range of from about 2 megarads (M.R.) to about 12 M.R. Any conventional cross-linking technique can be used. For example, electronic cross-linking may be performed using curtain-beam irradiation. Chemical cross-linking techniques can also be employed (e.g., by the use of peroxides).

In some embodiments, film 5 can be transparent (at least in the non-printed regions) such that the packaged product is visible through the film. The term “transparent” as used herein can refer to the ability of a material to transmit incident light with negligible scattering and little absorption, enabling objects (e.g., packaged food or print) to be seen clearly through the material under typical unaided viewing conditions (i.e., the expected use conditions of the material). The transparency of the film can be at least about any of the following values: 20%, 25%, 30%, 40%, 50%, 65%, 70%, 75%, 80%, 85%, and 95%, as measured in accordance with ASTM D1746. Alternatively, in some embodiments, film 5 can include one or more pigments (such as tan coloring), as can be appreciated by those of ordinary skill in the art. Further, in some embodiments, at least one surface of film 5 can be embossed or texturized to improve resistance to blocking, machinability, and/or handleability or to impart some performance benefit, such as softness or suppleness to the film.

In some embodiments, film 5 can comprise printed product information such as (but not limited to) product size, type, name of manufacturer, instructions for use, and the like. Such printing methods are well known to those of ordinary skill in the packaging art.

II.B. Odor Absorbing Layer

As set forth above, film 5 comprises at least one odor absorbing layer. More particularly, the odor absorbing layer comprises a polyvinylidene chloride polymer blended with a magnesium oxide, magnesium salt, magnesium hydroxide, and/or zeolite. The odor absorbing layer can be positioned as a core layer, an inner layer, a sealant layer, and/or an exterior layer of the film. The odor absorbing layer provides a barrier to gases as well as to odors. In some embodiments, the odor absorbing layer imparts to film 5 an average smell strength of about 3.0 or less/24 hours; in some embodiments, about 2.5 or less/24 hours; in some embodiments, about 2.0 or less/24 hours; in some embodiments, about 1.5 or less/24 hours; in some embodiments, about 1.0 or less/24 hours; and in some embodiments, about 0.5 or less/24 hours. Smell strength herein is measured in accordance with BS ISO 8670-3:2000, the entire content of which is hereby incorporated by reference.

As would be appreciated by those of ordinary skill in the art, the odor absorbing layer also imparts to film 5 an oxygen transmission rate of at most any of the following values: 150, 100, 50, 45, 40, 35, 30, 25, 20, 15, 10, and 5 cubic centimeters (at standard temperature and pressure) per square meter per day per 1 atmosphere of oxygen pressure differential (50 cm³/m²-d-atm) measured at 0% relative humidity and 23° C. Thus, in some embodiments, the oxygen permeability of film 5 can be below about 50 cm³/m²-d-atm; in some embodiments, below about 40 cm³/m²-d-atm; in some embodiments, below about 30 cm³/m²-d-atm; and in some embodiments, below about 20 cm³/m²-d-atm at 23° C. and 0% relative humidity. All references to oxygen transmission rate herein are measured at these conditions in accordance with ASTM D-3985, the entire content of which is hereby incorporated by reference.

The presently disclosed film includes an odor absorbent layer comprising polyvinylidene chloride polymer blended with a magnesium oxide, magnesium salt, magnesium hydroxide, and/or a zeolite. Vinylidene chloride polymers (“PVDC”), such as vinylidene chloride/methyl acrylate copolymer (VDC/MA), refer to a vinylidene chloride-containing polymer or copolymer. That is, a polymer that includes monomer units derived from vinylidene chloride (CH₂[H]CCl₂) and monomer units derived from one or more of the following: methyl acrylate, vinyl chloride, styrene, vinyl acetate, and acrylonitrile. Thus, for example, film 5 can comprise in some embodiments at least one layer that includes a PVDC/MA copolymer.

In some embodiments, PVDC copolymer suitable for use with the presently disclosed subject matter can have between about 85 weight % and 99 weight % PVDC monomer. Thus, the PVDC copolymer of the odor absorbing layer can comprise about 85, 86, 87, 88, 89, 90, 91, 92, 93, 94, 95, 96, 97, 98, or 99 weight percent PVDC monomer. To this end, the odor absorbing layer can comprise about 1, 2, 3, 4, 5, 6, 7, 8, 9, 10, 11, 12, 13, 14, or 15 weight percent comonomer (such as methyl acrylate, for example).

In some embodiments, the polyvinylidene chloride polymer can be present in the odor absorbing layer of film 5 in an amount of from about 85% to about 99.9%; in some embodiments, from about 90% to about 98%; and in some embodiments, from about 95% to about 97%, based on the total weight of the layer. Thus, the PVDC can be present in an amount of from about 85, 85.5, 90, 90.5, 91, 91.5, 92, 92.5, 93, 93.5, 94, 94.5, 95, 95.5, 96, 96.5, 97, 97.5, 98, 98.5, 99, 99.5, or 99.9%, based on the total weight of the layer.

Suitable PVDC polymers are available from Dow Chemical under the tradename SARAN® (e.g., SARAN® MA127, SARAN® XU32019.10, XU32937) and from Solvay® (e.g., IXAN PV891, PV910, and PV925). However, the presently disclosed subject matter can include any of a wide variety of PVDC polymers and is not limited to those set forth above.

Continuing, the odor absorbing layer can comprise magnesium oxide, magnesium salt, and/or magnesium hydroxide. Suitable magnesium oxide compositions are available commercially as, for example, Elastomag® 100, Elastomag® 170 MgO, Elastomag® 170 MgO Special, and Elastomag® FE MgO (Martin Marietta Magnesia Specialties, Baltimore, Md., United States of America). Suitable magnesium hydroxide compositions are available commercially as, for example, DHT-4A®, DHT-4V®, and DHT-4A-2® (Mitsui Petrochemical Corporation, New York, N.Y., United States of America). It has been surprisingly discovered that the incorporation of a magnesium oxide, magnesium hydroxide, and/or magnesium salt into the odor absorbing layer of film 5 improves the film's odor absorbing capabilities without affecting the barrier properties of the film.

In some embodiments, the magnesium oxide, magnesium salt, and/or magnesium hydroxide can be present in the odor absorbing layer of film 5 in an amount of from about 0.1% to about 10%; in some embodiments, from about 1% to about 9%; and in some embodiments, from about 2% to about 8%, based on the total weight of the layer. Thus, the magnesium oxide can be present in an amount of from about 0.1, 0.5, 1, 1.5, 2, 2.5, 3, 3.5, 4, 4.5, 5, 5.5, 6, 6.5, 7, 7.5, 8, 8.5, 9, 9.5, or 10%, based on the total weight of the layer.

Film 5 includes an odor absorbent layer comprising a polyvinylidene chloride polymer bended with a magnesium oxide, magnesium salt, magnesium hydroxide, and/or a zeolite. Zeolites are known in the art and are generally characterized by an aluminosilicate tetrahedral framework and have ion exchangeable large cations and loosely held water molecules permitting reversible dehydration. The general formula for a zeolite is MO.Al₂O₃.nSiO.xH₂O, where M is Na, K, Ca, Sr, or Ba and n and x are integers. Zeolites suitable for use in film 5 can include naturally occurring or synthetic zeolites.

To this end, polyvinylidene chloride polymer can be blended with any suitable naturally occurring zeolite. For example, such naturally occurring zeolites can include, but are not limited to, one or more of the following: analcime, wairakite, pollucite, sodalite, faujasite, chabazite, gmelinite, erionite, offretite, levyne, natrolite, scolecite, mesolite, edingtonite, thomsonite, gonnardite, harmontome, gismondine, garronite, mordenite, diachiardite, clinoptilolite, heulandite, brewsterite, epistilbite, stilbite, yugawaralite, laumontite, ferrierite, paulingite. See, for example, U.S. Pat. No. 6,284,232, the entire disclosure of which is hereby incorporated by reference.

Alternatively or in addition, in the presently disclosed subject matter polyvinylidene chloride polymer can be blended with any suitable synthetic zeolite. For example, such suitable synthetic zeolites can include, but are not limited to, one or more of the following: zeolite A, zeolite X, zeolite, Y, zeolite ZK-5, zeolite ZK-4, zeolite ZSM-5, zeolite ZSM-1, zeolite ZSM-12, zeolite ZSM-20, zeolite ZSM-23, zeolite ZSM-35, and zeolite ZSM-38. See, for example, U.S. Pat. Nos. 2,882,243; 2,882,244; 3,130,007; 3,247,195; 3,314,752; 3,702,886; 3,709,979; 3,832,449; 3,972,983; 4,075,842; 4,016,245; 4,046,859, the entire disclosures of which are hereby incorporated by reference.

Thus, any of a wide variety of zeolites can be used to prepare film 5. For example, commercially available zeolite suitable for use in the presently disclosed subject matter can include (but are not limited to) Abscents 3000 (available commercially from UOP Laboratories, Des Plaines, Ill., United States of America) and ChemPlasa® OAB-101, ChemPlasa® OAB-102, ChemPlasa® OAB-104 (available from ChemPlasa, Changzhou, Jiangsu, China). Without being bound by any particular theory, it is believed that the zeolite molecules are capable of absorbing odors from an atmosphere (such as, for example, the interior of an ostomy pouch).

In some embodiments, the zeolite can be present in the odor absorbing layer of film 5 in an amount of from about 0.1% to about 10%; in some embodiments, from about 0.2% to about 8%; and in some embodiments, from about 0.3% to about 5%, based on the total weight of the layer. Thus, the zeolite can be present in an amount of from about 0.1, 0.5, 1, 1.5, 2, 2.5, 3, 3.5, 4, 4.5, 5, 5.5, 6, 6.5, 7, 7.5, 8, 8.5, 9, 9.5, or 10%, based on the total weight of the layer.

In addition to the polyvinylidene chloride polymer, magnesium oxide, magnesium hydroxide, magnesium salt, and/or zeolite, the odor absorbent layer of film 5 can optionally include one or more additives. For example, suitable additives can include (but are not limited to) thermal stabilizers (e.g., epoxidized soybean oil), antiblock agents (such as fluoropolymer), lubricating processing aids (e.g., one or more acrylates, such as Metablen L1000™, available from Elf Atochemicals, Philadelphia, Pa., United States of America), processing aids, slip agents, pigments, and the like. Such additives are well known to those of ordinary skill in the packaging art.

II.C. Additional Layers of Film 5

As set forth herein above, film 5 can include one or more layers in addition to the odor absorbing layer. For example, in some embodiments, the disclosed film can include one or more of each of the following: a product-side or inside layer (i.e., a heat seal layer), a non-product or outside layer (i.e., a print side layer), a gas barrier layer, a tie layer, an abuse layer, and a bulk layer. Below are some examples of preferred combinations in which the alphabetical symbols designate the resin layers. Where the multilayer film representation below includes the same letter more than once, each occurrence of the letter may represent the same composition or a different composition within the class that performs a similar function.

A/D, A/C/D, A/B/D, A/B/C/D, A/C/B/D, A/B/C/E/D, A/E/C/E/D, A/B/E/C/D, A/C/B/E/D, A/C/E/B/D, A/E/B/C/D, A/E/C/B/D, A/C/B/C/D, A/B/C/B/D, A/B/C/E/B/D, A/B/C/E/C/D, A/B/E/C/B/D, A/C/E/C/B/D, A/B/C/B/B/D, A/C/B/B/B/D, A/C/B/C/B/D, A/C/E/B/B/D, A/B/E/C/E/B/D, A/B/E/C/E/B/E/D, where “A” is the inside layer, heat seal layer, or food-side layer, as discussed below. “B” is a core or bulk layer, as discussed below. “C” is a barrier layer, as discussed below. “D” is an outside (print) layer, as discussed below. “E” is a tie layer, as discussed below. The odor absorbing layer as discussed herein can be the core or bulk layer (“B”) or the barrier layer (“C”).

One or more layers of the disclosed film can include one or more additives useful in packaging films, such as (but not limited to) antifog agents, antiblocking agents, slip agents, colorants, pigments, dyes, flavorants, antimicrobial agents, preservatives, antioxidants, fillers, radiation stabilizers, antistatic agents, and the like. Such additives and their effective amounts are known in the art.

### III. Methods of Making Film 5

The disclosed film can be manufactured by any of a variety of processes known in the art, including extrusion (e.g., blow-film extrusion, coextrusion, extrusion coating, free film extrusion, and lamination), casting, and adhesive lamination. A combination of these processes can also be employed. Such processes are well known to those of ordinary skill in the packaging art. For example, extrusion coating is described in U.S. Pat. No. 4,278,738 to Brax, the entire content of which is incorporated herein by reference. Coextrusion manufacture can use, for example, a tubular trapped bubble film process or a flat film (i.e., cast film or slit die) process.

### IV. Methods of Using Film 5

When film 5 is used to form a pouch (such as an ostomy pouch), external layer 35 in some embodiments forms the outer surface of the pouch (i.e., the surface that is exposed to the environment) while seal layer 30 forms the inner surface of the pouch (i.e., the surface which is in contact with the inside of the pouch and, therefore, with the product, drainage, or air that is enclosed within the pouch). In this role, peripheral portions of the seal layer can be joined, e.g., by heat-sealing, impulse-sealing, or radio frequency (RF) sealing, to form an enclosure.

In the case of heat-sealing, the sealant layer is either folded upon itself or mated with the sealant layer of another piece of film such that two regions of the sealant layer are brought into contact with one another and sufficient heat is applied to predetermined, e.g., peripheral, segments of the contacting regions of the second exterior layer that the heated segments become molten and intermix with one another. Upon cooling, the heated segments of the second exterior layer become a single, essentially inseparable layer. In this manner, the heated segments of the second exterior layer produce a liquid-tight closure which is commonly referred to as a heat seal. The heat seals thus formed are linked together to define the peripheral boundaries of the pouch so that the pouch contents can be fully enclosed therein. Accordingly, the disclosed film structure can advantageously be used to form a drainage pouch, such as an ostomy pouch. Such a structure combines excellent gas-barrier functionality and strength with a high degree of odor barrier.

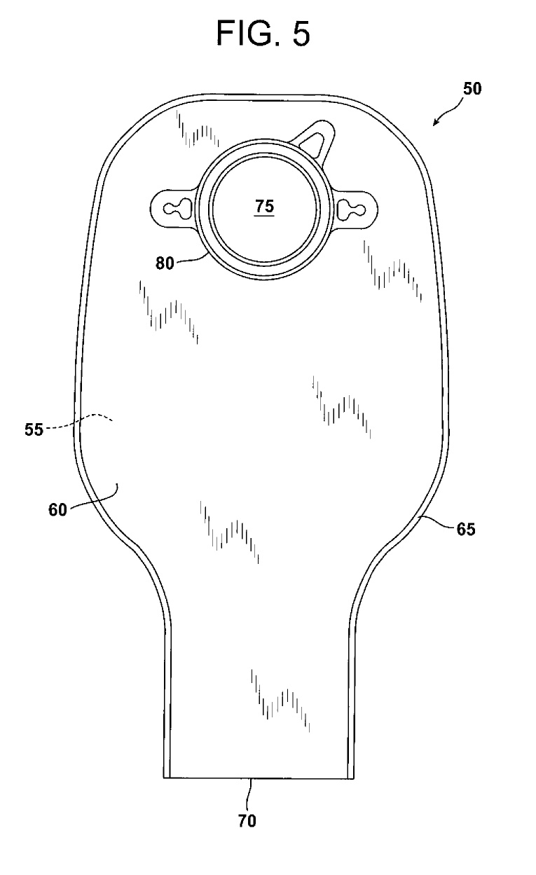

FIG. 5 illustrates one embodiment of an ostomy pouch that can be constructed from the presently disclosed film. Particularly, pouch 50 comprises front and rear walls 55, 60 joined along their edges by heat seal 65 or any other suitable means. The pouch has a drain opening 70 that can be closed by folding, clamping, and/or any of a wide variety of known closure techniques. In some embodiments, rear wall 60 includes a stoma-receiving opening 75 surrounded by attachment 80. In the embodiment depicted in FIG. 5, the pouch is one component of a two-piece appliance and its attachment can take the form of a coupling ring having a channel for releasably engaging the mating element of a faceplate coupling ring (not shown), all of which is known in the art. Alternatively, attachment 80 can take the form of an adhesive ring or patch designed to adhesively engage the peristomal skin surfaces of a wearer (i.e., a one-piece appliance) or the smooth surface of a faceplate that is adhesively secured to the wearer (an adhesive two-piece appliance). To this end, other layers can be added to film 5, such as a comfort ply of moisture absorbent or waterproof tissue paper to improve the feel on the skin of the wearer. The ostomy pouch depicted in FIG. 5 is one of a wide variety of pouches that can be formed from film 5. As such, the ostomy pouch of FIG. 5 is in no way intended to be limiting.

In use, when film 5 is formed into pouch 50, as odors and gases accumulate within the pouch interior, they are absorbed into the odor absorbing layer of the film. As a result, the odors become trapped in the film and do not pass out of the pouch.

The disclosed films have been described in connection with medical applications. However, it is to be understood that other applications for the films are also possible, and that this disclosure should not be construed as being limited only to medical pouches or devices.

### V. Benefits of the Presently Disclosed Subject Matter

The presently disclosed film offers a superior combination of properties, i.e., the advantage of being a good barrier to both gases and odors. Specifically, the disclosed film includes one or more additives that provides the film with enhanced odor absorbing capabilities, while also maintaining the effective gas barrier properties.

The disclosed films also have high noiselessness and pliability characteristics, which render the films suitable for a wide variety of applications. For example, the films can be particularly suitable for the manufacture of containers and bags intended for human drainage in medical applications (i.e., ostomy/urostomy use).

One of ordinary skill in the art would recognize that the benefits described herein above are non-limiting. To this end, the presently disclosed subject matter can include benefits not specified above.

### EXAMPLES

The following examples provide illustrative embodiments. In light of the present disclosure and the general level of skill in the art, those of ordinary skill can appreciate that the following examples are intended to be exemplary only and that numerous changes, modifications, and alterations can be employed without departing from the scope of the presently claimed subject matter.

Several film structures and comparatives are identified herein below.

TABLE 1
Resin Identification

| Material | Trade name Or | |
|---|---|---|
| Code | Designation | Source(s) |
| A | SYLOBLOC 47 | GRACE Davison |
| | | (Deerfield, Illinois, United |
| | | States of America) |
| B | Kemamide E Pellet | PMC Biogenix, Inc. (Mt. |
| | | Laurel, New Jersey, |
| | | United States of America) |
| C | ELVAX 3165 | DuPont Dow Elastomers |
| | | (Wilmington, Delaware, |
| | | United States of America) |
| D | Elvax 3165VLGA | DuPont Dow Elastomers |
| | | (Wilmington, Delaware, |
| | | United States of America) |
| E | EVATANE 28-03 | Arkema (Colombes, |
| | | France) |
| F | IXAN PV910 | Solvay Plastics |
| | | (Brussels, Belgium) |
| G | DHT-4A | Mitsui Petrochemical |
| | | Corporation (New York, |
| | | New York, United States |
| | | of America) |
| H | Dehysol D 82 | IMCD Deutschland |
| I | SABIC LLDPE 118N | SABIC (Riyadh, Saudi |
| | | Arabia) |
| J | ampacet 102121 | Ampacet (Tarrytown, |
| | | New York, United States |
| | | of America) |
| K | AFFINITY EG 8100 | Dow Chemical Company |
| | | (Midland, Michigan, |
| | | United States of America) |
| L | XVS117 | Solvay Plastics |
| | | (Brussels, Belgium) |
| M | PVS118 | Solvay Plastics |
| | | (Brussels, Belgium) |
| N | LVS119 | Solvay Plastics |
| | | (Brussels, Belgium) |
| O | FIBAPLAST BEIGE EVA | Karl Finke GmbH & Co. |
| | 440493 | KG (Wuppertal, |
| | | Germany) |
| P | AFFINITY EG 8100 | Dow Chemical Company |
| | | (Midland, Michigan, |
| | | United States of America) |
| Q | IXAN PVS118 | Solvay Plastics |
| | | (Brussels, Belgium) |
| R | TN2006 | Braskem S.A. (Sao |
| | | Paulo, Brazil) |
| S | CESA PV82050202-ZN | Clariant Corporation |
| | | (Charlotte, North |
| | | Carolina, United States of |
| | | America) |
| T | LF1020AA | Westlake Chemical |
| | | Corporation (Houston, |
| | | Texas, United States of |
| | | America) |
| U | PV73025553 | Clariant Corporation |
| | | (Charlotte, North |
| | | Carolina, United States of |
| | | America) |
| V | IXAN PV925 | Solvay Plastics |
| | | (Brussels, Belgium) |
| W | PLASTISTRENGTH | Arkema (Colombes, |
| | L1000 | France) |
| X | OAB-101 | ChemPlasa (Changzhou, |
| | | Jiangsu, China) |

A is antiblock.
B is slip agent with DSC melting point of 81-86° C. and a moisture content of 0.2% maximum.
C is ethylene/vinyl acetate copolymer with flow rate of 0.6-0.8 g/10 min, vinyl acetate content of 17.4-18.6%, and density of 0.94 g/cc.
D is ethylene/vinyl acetate copolymer with flow rate of 0.6-0.8 g/10 min, vinyl acetate content of 17.4-18.6%, and density of 0.94 g/cc.
E is ethylene/vinyl acetate copolymer (28 weight % VA, 3 mil).
F is vinylidene chloride/methyl acrylate copolymer (8.5 weight % MA).
G is magnesium aluminum hydroxy carbonate hydrate with specific gravity of 2.1 g/cc.
H is epoxidized soybean oil.
I is linear low density ethylene/butene copolymer.
J is antiblock and slip with flow rate of 1.1-2.5 g/10 minutes and specific gravity of 0.94-0.96.
K is very low density ethylene/octene copolymer with flow rate of 0.75-1.25 g/10 min and density of 0.867-0.873 g/cc.
L is vinylidene chloride/methyl acrylate copolymer (8.0 weight % MA).
M is vinylidene chloride/methyl acrylate copolymer (8.0 weight % MA).
N is vinylidene chloride/methyl acrylate copolymer (8.0 weight % MA).
O is beige color concentrate in ethylene/vinyl acetate copolymer.
P is branched, single site very low density ethylene/octene copolymer with flow rate (condition E) of 0.75-1.25 g/10 min. and density of 0.867-0.873 g/cc.
Q is vinylidene chloride/methyl acrylate copolymer.
R is ethylene/vinyl acetate copolymer (10-20 wt % comonomer).
S is antibock and slip in ethylene/vinyl acetate copolymer.
T is linear low density ethylene/butene copolymer.
U is grey color concentrate in low density polyethylene.
V is vinylidene chloride/methyl acrylate copolymer (8.0 weight % MA).
W is butyl acrylate/methyl methacrylate/butyl methacrylate terpolymer with density of 0.25-0.50 g/mL, specific gravity of 1.13-1.15, and melting range of 150-160° C.
X is zeolite with BET surface area (m²/g) of 300 min and particle size of 4 μm.

TABLE 2
Film Identification

| | | | | Layer |
|---|---|---|---|---|
| | | | | Thickness |
| Film ID | Layer | Formulation | Volume % | (mils) |
| Film 1 | 1 | 95.5% D | 32.7 | 0.96 |
| | | 3.6% C | | |
| | | 0.54% B | | |
| | | 0.36% A | | |
| | 2 | 100% E | 13.3 | 0.39 |
| | 3 | 97% F | 8.0 | 0.24 |
| | | 2% H | | |
| | | 1% G | | |
| | 4 | 100% E | 13.3 | 0.39 |
| | 5 | 96% D | 32.7 | 0.96 |
| | | 3.2% C | | |
| | | 0.48% B | | |
| | | 0.32% A | | |
| Film 2 | 1 | 70% D | 9.3 | 0.28 |
| | | 27% I | | |
| | | 3% J | | |
| | 2 | 50% D | 29.3 | 0.87 |
| | | 25% I | | |
| | | 25% K | | |
| | 3 | 100% E | 6.7 | 0.20 |
| | 4 | 100% L | 9.3 | 0.28 |
| | 5 | 100% E | 6.7 | 0.20 |
| | 6 | 50% D | 29.3 | 0.87 |
| | | 25% I | | |
| | | 25% K | | |
| | 7 | 70% D | 9.3 | 0.28 |
| | | 27% I | | |
| | | 3% J | | |
| Film 3 | 1 | 70% D | 9.4 | 0.28 |
| | | 27% I | | |
| | | 3% J | | |
| | 2 | 50% D | 29.2 | 0.87 |
| | | 25% I | | |
| | | 25% K | | |
| | 3 | 100% E | 6.7 | 0.20 |
| | 4 | 100% M | 9.4 | 0.28 |
| | 5 | 100% E | 6.7 | 0.20 |
| | 6 | 50% D | 29.2 | 0.87 |
| | | 25% I | | |
| | | 25% K | | |
| | 7 | 70% D | 9.4 | 0.28 |
| | | 27% I | | |
| | | 3% J | | |
| Film 4 | 1 | 70% D | 9.4 | 0.28 |
| | | 27% I | | |
| | | 3% J | | |
| | 2 | 50% D | 29.2 | 0.87 |
| | | 25% I | | |
| | | 25% K | | |
| | 3 | 100% E | 6.7 | 0.20 |
| | 4 | 100% N | 9.4 | 0.28 |
| | 5 | 100% E | 6.7 | 0.20 |
| | 6 | 50% D | 29.2 | 0.87 |
| | | 25% I | | |
| | | 25% K | | |
| | 7 | 70% D | 9.4 | 0.28 |
| | | 27% I | | |
| | | 3% J | | |
| Film 5 | 1 | 95.5% D | 32.7 | 0.96 |
| | | 3.6% C | | |
| | | 0.54% B | | |
| | | 0.36% A | | |
| | 2 | 100% E | 13.3 | 0.39 |
| | 3 | 97% F | 8.0 | 0.24 |
| | | 2% H | | |
| | | 1% G | | |
| | 4 | 100% E | 13.3 | 0.39 |
| | 5 | 96% D | 32.7 | 0.96 |
| | | 3.2% C | | |
| | | 0.48% B | | |
| | | 0.32% A | | |
| Film 6 | 1 | 70% R | 9.4 | 0.28 |
| | | 26% Q | | |
| | | 0.3% A | | |
| | | 0.5% B | | |
| | | 3.2% C | | |
| | 2 | 50% R | 29.2 | 0.87 |
| | | 25% I | | |
| | | 25% P | | |
| | 3 | 100% E | 6.7 | 0.20 |
| | 4 | 100% Q | 9.4 | 0.28 |
| | 5 | 100% E | 6.7 | 0.20 |
| | 6 | 50% R | 29.2 | 0.87 |
| | | 25% I | | |
| | | 25% P | | |
| | 7 | 70% R | 9.4 | 0.28 |
| | | 26% Q | | |
| | | 0.3% A | | |
| | | 0.5% B | | |
| | | 3.2% C | | |
| Film 7 | 1 | 67% R | 9.4 | 0.28 |
| | | 26% I | | |
| | | 0.3% A | | |
| | | 0.5% B | | |
| | | 3.2% C | | |
| | | 3% O | | |
| | 2 | 47% R | 29.2 | 0.87 |
| | | 25% I | | |
| | | 25% P | | |
| | | 3% O | | |
| | 3 | 100% E | 6.7 | 0.20 |
| | 4 | 100% Q | 9.4 | 0.28 |
| | 5 | 100% E | 6.7 | 0.20 |
| | 6 | 47% R | 29.2 | 0.87 |
| | | 25% I | | |
| | | 25% P | | |
| | | 3% O | | |
| | 7 | 67% R | 9.4 | 0.28 |
| | | 26% I | | |
| | | 0.3% A | | |
| | | 0.5% B | | |
| | | 3.2% C | | |
| | | 3% O | | |
| Film 8 | 1 | 53% R | 9.4 | 0.28 |
| | | 20% S | | |
| | | 27% I | | |
| | 2 | 35% R | 29.2 | 0.87 |
| | | 25% I | | |
| | | 25% P | | |
| | | 26% S | | |
| | 3 | 100% E | 6.7 | 0.20 |
| | 4 | 100% Q | 9.4 | 0.28 |
| | 5 | 100% E | 6.7 | 0.20 |
| | 6 | 35% R | 29.2 | 0.87 |
| | | 25% I | | |
| | | 25% P | | |
| | | 26% S | | |
| | 7 | 53% R | 9.4 | 0.28 |
| | | 20% S | | |
| | | 27% I | | |
| Film 9 | 1 | 70% R | 9.4 | 0.28 |
| | | 26% T | | |
| | | 0.3% A | | |
| | | 0.5% B | | |
| | | 3.2% C | | |
| | 2 | 50% R | 29.2 | 0.87 |
| | | 25% T | | |
| | | 25% P | | |
| | 3 | 100% E | 6.7 | 0.20 |
| | 4 | 100% Q | 9.4 | 0.28 |
| | 5 | 100% E | 6.7 | 0.20 |
| | 6 | 50% R | 29.2 | 0.87 |
| | | 25% T | | |
| | | 25% P | | |
| | 7 | 70% R | 9.4 | 0.28 |
| | | 26% T | | |
| | | 0.3% A | | |
| | | 0.5% B | | |
| | | 3.2% C | | |
| Film 10 | 1 | 67% R | 9.4 | 0.28 |
| | | 26% T | | |
| | | 0.3% A | | |
| | | 0.5% B | | |
| | | 3.2% C | | |
| | | 3% O | | |
| | 2 | 47% R | 29.2 | 0.87 |
| | | 25% T | | |
| | | 25% P | | |
| | | 3% O | | |
| | 3 | 100% E | 6.7 | 0.20 |
| | 4 | 100% Q | 9.4 | 0.28 |
| | 5 | 100% E | 6.7 | 0.20 |
| | 6 | 47% R | 29.2 | 0.87 |
| | | 25% T | | |
| | | 25% P | | |
| | | 3% O | | |
| | 7 | 67% R | 9.4 | 0.28 |
| | | 26% T | | |
| | | 0.3% A | | |
| | | 0.5% B | | |
| | | 3.2% C | | |
| | | 3% O | | |
| Film 11 | 1 | 53% R | 9.4 | 0.28 |
| | | 27% T | | |
| | | 20% S | | |
| | 2 | 35% R | 29.2 | 0.87 |
| | | 25% T | | |
| | | 25% P | | |
| | | 15% S | | |
| | 3 | 100% E | 6.7 | 0.20 |
| | 4 | 100% Q | 9.4 | 0.28 |
| | 5 | 100% E | 6.7 | 0.20 |
| | 6 | 35% R | 29.2 | 0.87 |
| | | 25% T | | |
| | | 25% P | | |
| | | 15% S | | |
| | 7 | 53% R | 9.4 | 0.28 |
| | | 27% T | | |
| | | 20% S | | |
| Film 12 | 1 | 67% R | 9.4 | 0.28 |
| | | 26% T | | |
| | | 0.3% A | | |
| | | 0.5% B | | |
| | | 3.2% C | | |
| | | 3% U | | |
| | 2 | 47% R | 29.2 | 0.87 |
| | | 25% T | | |
| | | 25% P | | |
| | | 3% U | | |
| | 3 | 100% E | 6.7 | 0.20 |
| | 4 | 100% Q | 9.4 | 0.28 |
| | 5 | 100% E | 6.7 | 0.20 |
| | 6 | 47% R | 29.2 | 0.87 |
| | | 25% T | | |
| | | 25% P | | |
| | | 3% U | | |
| | 7 | 67% R | 9.4 | 0.28 |
| | | 26% T | | |
| | | 0.3% A | | |
| | | 0.5% B | | |
| | | 3.2% C | | |
| | | 3% U | | |
| Film 13 | 1 | 70% R | 9.5 | 0.24 |
| | | 26% T | | |
| | | 0.3% A | | |
| | | 0.5% B | | |
| | | 3.2% C | | |
| | 2 | 50% R | 29.4 | 0.73 |
| | | 25% T | | |
| | | 25% P | | |
| | 3 | 100% E | 6.3 | 0.16 |
| | 4 | 100% Q | 9.5 | 0.37 |
| | 5 | 100% E | 6.3 | 0.16 |
| | 6 | 50% R | 29.4 | 0.73 |
| | | 25% | | |
| | | 25% P | | |
| | 7 | 70% R | 9.5 | 0.24 |
| | | 26% T | | |
| | | 0.3% A | | |
| | | 0.5% B | | |
| | | 3.2% C | | |
| Film 14 | 1 | 70% R | 9.0 | 0.18 |
| | | 26% T | | |
| | | 0.3% A | | |
| | | 0.5% B | | |
| | | 3.2% C | | |
| | 2 | 50% R | 28.0 | 1.10 |
| | | 25% T | | |
| | | 25% P | | |
| | 3 | 100% E | 8.0 | 0.31 |
| | 4 | 100% Q | 10.0 | 0.39 |
| | 5 | 100% E | 8.0 | 0.31 |
| | 6 | 50% R | 28.0 | 1.10 |
| | | 25% | | |
| | | 25% P | | |
| | 7 | 70% R | 9.0 | 0.18 |
| | | 26% T | | |
| | | 0.3% A | | |
| | | 0.5% B | | |
| | | 3.2% C | | |
| Film 15 | 1 | 67% R | 9.5 | 0.24 |
| | | 26% T | | |
| | | 0.3% A | | |
| | | 0.5% B | | |
| | | 3.2% C | | |
| | | 3% O | | |
| | 2 | 47% R | 29.4 | 0.73 |
| | | 25% T | | |
| | | 25% P | | |
| | | 3% O | | |
| | 3 | 100% E | 6.3 | 0.16 |
| | 4 | 100% Q | 9.5 | 0.24 |
| | 5 | 100% E | 6.3 | 0.16 |
| | 6 | 47% R | 29.4 | 0.73 |
| | | 25% T | | |
| | | 25% P | | |
| | | 3% O | | |
| | 7 | 67% R | 9.5 | 0.24 |
| | | 26% T | | |
| | | 0.3% A | | |
| | | 0.5% B | | |
| | | 3.2% C | | |
| | | 3% O | | |
| Film 16 | 1 | 67% R | 9.0 | 0.18 |
| | | 26% T | | |
| | | 0.3% A | | |
| | | 0.5% B | | |
| | | 3.2% C | | |
| | | 3% O | | |
| | 2 | 47% R | 28.0 | 0.55 |
| | | 25% T | | |
| | | 25% P | | |
| | | 3% O | | |
| | 3 | 100% E | 8.0 | 0.16 |
| | 4 | 100% Q | 10.0 | 0.20 |
| | 5 | 100% E | 8.0 | 0.16 |
| | 6 | 47% R | 28.0 | 0.55 |
| | | 25% T | | |
| | | 25% P | | |
| | | 3% O | | |
| | 7 | 67% R | 9.0 | 0.18 |
| | | 26% T | | |
| | | 0.3% A | | |
| | | 0.5% B | | |
| | | 3.2% C | | |
| | | 3% O | | |
| Film 17 | 1 | 100% C | 34.0 | 0.45 |
| | 2 | 100% V | 7.0 | 0.10 |
| | 3 | 100% C | 34.0 | 0.45 |
| Film 18 | 1 | 100% C | 34.0 | 0.45 |
| | 2 | 98.7% V | 7.0 | 0.10 |
| | | 1% G | | |
| | | 0.3% H | | |
| | 3 | 100% C | 34.0 | 0.45 |
| Film 19 | 1 | 100% C | 34.0 | 0.45 |
| | 2 | 98.4% V | 7.0 | 0.10 |
| | | 1% G | | |
| | | 0.6% O | | |
| | 3 | 100% C | 34.0 | 0.45 |
| Film 20 | 1 | 67% D | 9.4 | 0.28 |
| | | 26% I | | |
| | | 3% O | | |
| | | 3.2% C | | |
| | | 0.48% B | | |
| | | 0.32% A | | |
| | 2 | 47% D | 29.2 | 0.87 |
| | | 25% I | | |
| | | 25% K | | |
| | | 3% O | | |
| | 3 | 100% E | 6.7 | 0.20 |
| | 4 | 100% Q | 9.4 | 0.28 |
| | 5 | 100% E | 6.7 | 0.20 |
| | 6 | 47% D | 29.2 | 0.87 |
| | | 25% I | | |
| | | 25% K | | |
| | | 3% O | | |
| | 7 | 67% D | 9.4 | 0.28 |
| | | 26% I | | |
| | | 3% O | | |
| | | 3.2% C | | |
| | | 0.48% B | | |
| | | 0.32% A | | |

### Example 1

### Preparation of Films 1-5

Films 1-5 were manufactured by coextrusion. This method is well known to those of ordinary skill in the art.

### Example 2

### Preparation of Ostomy Bags 1-5

Films 1-5 were used to prepare ostomy bags 1-5, respectively, without filters or other attachments according to standard techniques well known in the packaging art.

### Example 3

### Odor Transmission of Ostomy Bags 1-4

A medium-sized onion was chopped into pieces less than 5 mm in size. The chopped onions were wrapped in aluminum foil and stored at room temperature for no longer than 1 hour.

About 20 g of the chopped onion was placed inside each ostomy bag 1-4 and positioned such that none of the onion came into contact with the outer surface of the ostomy bag or with the ostomy bag flange. The openings of ostomy bags 1-4 were then sealed using a metal, plastic, or glass plate, ensuring that there were no creases that could create leaks.

About 50 mL of water was added to the bottom of 4 glass jars with a volume of about 2 L. An upturned glass beaker was positioned on the bottom of each glass jar to form a platform. Ostomy bag 1 was placed on the platform of glass jar 1 and positioned so that the bag was not in contact with the water. Similarly, ostomy bags 2-4 were placed on the platforms of glass jars 2-4, respectively. The glass jars were then sealed and placed in a water bath at a temperature of about 34° C.

Five people with a normal sense of smell (“sniffers”) were selected. At 0.5, 1, 2, 4, 6, 8, and 24 hour timepoints, the lid was removed from each jar and the first sniffer was allowed to sniff the atmosphere inside the jar. After a maximum time of 5 seconds, the jar was closed. After at least 1 minute, the lid was again removed and the second sniffer was allowed to sniff the atmosphere inside the jar. The procedure was repeated until each of the five sniffers sampled the atmosphere inside the jar once. Odor intensity was evaluated by means of the panel of sniffers using an odor scoring scale. The scores range from 0=none (i.e., excellent barrier properties) to 4=strong odor (i.e., poor barrier performance).

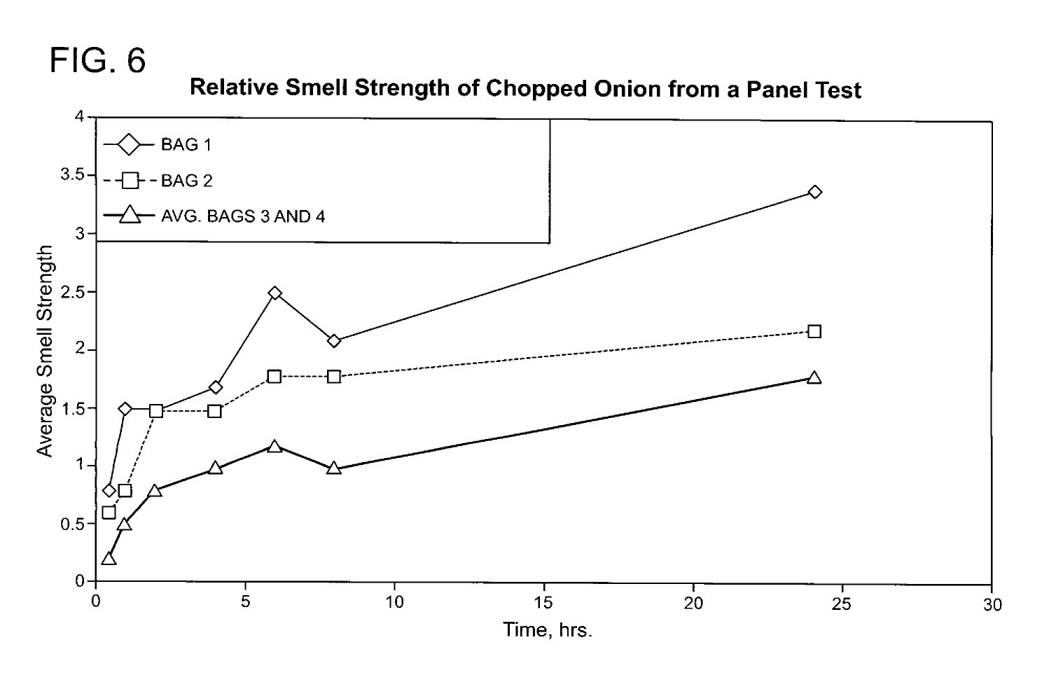

The results of the test are given below in Table 3 and are depicted in FIG. 6. The data from Ostomy Bags 3 and 4 were averaged. Each score reported in Table 3 is the average of the evaluation given by the 5 panelists.

TABLE 3
Odor Transmission of Ostomy Bags 1-4

| | Odor Transmission | | |
|---|---|---|---|
| | | | Average Ostomy |
| Time (Hours) | Ostomy Bag 1 | Ostomy Bag 2 | Bags 3 and 4 |
| 0.5 | 0.8 | 0.6 | 0.2 |
| 1 | 1.5 | 0.8 | 0.5 |
| 2 | 1.5 | 1.5 | 0.8 |
| 4 | 1.7 | 1.5 | 1.0 |
| 6 | 2.5 | 1.8 | 1.2 |
| 8 | 2.1 | 1.8 | 1.0 |
| 24 | 3.4 | 2.2 | 1.8 |

### Example 4

### Odor Transmission of Ostomy Bags 2, 3, 4, and 5

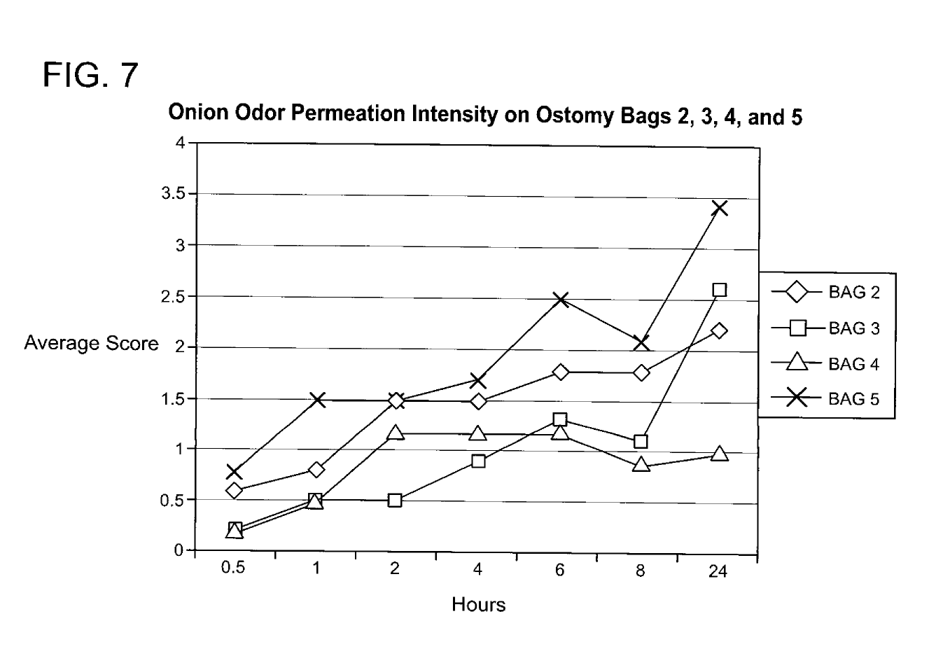

The odor barrier test of Example 3 was repeated using Bags 2, 3, 4, and 5. The results are provided below in Table 4 and FIG. 7.

TABLE 4
Odor Transmission of Ostomy Bags 2, 3, 4, and 5

| | Odor Transmission | | | |
|---|---|---|---|---|
| Time | | | | Ostomy |
| (Hours) | Ostomy Bag 2 | Ostomy Bag 3 | Ostomy Bag 4 | Bag 5 |
| 0.5 | 0.6 | 0.2 | 0.2 | 0.8 |
| 1 | 0.8 | 0.5 | 0.5 | 1.5 |
| 2 | 1.5 | 0.5 | 1.2 | 1.5 |
| 4 | 1.5 | 0.9 | 1.2 | 1.7 |
| 6 | 1.8 | 1.3 | 1.2 | 2.5 |
| 8 | 1.8 | 1.1 | 0.9 | 2.1 |
| 24 | 2.2 | 2.6 | 1.0 | 3.4 |

### Example 5

### Oxygen Transmission Rate of Films 1-4 and 6-12

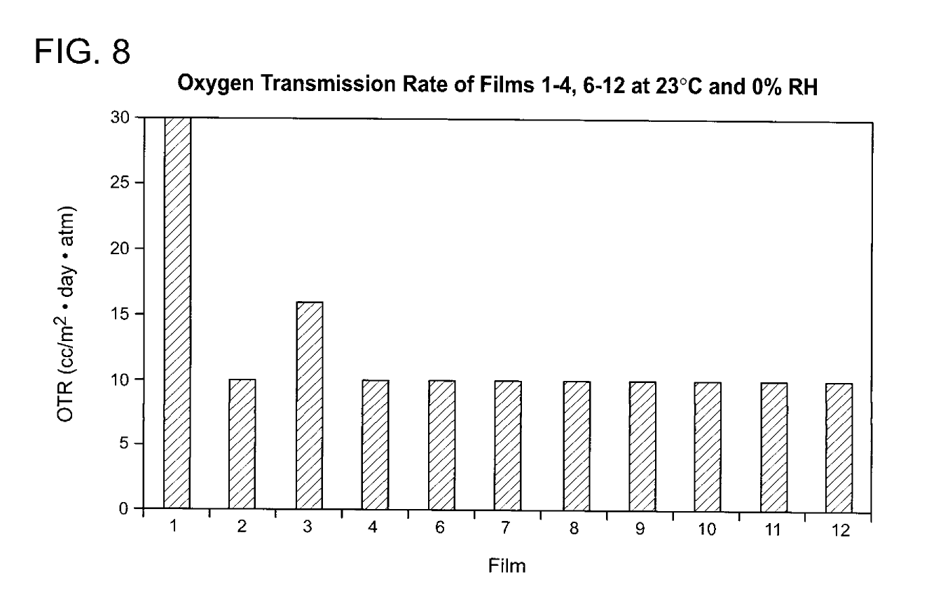

Evaluation of the oxygen transmission rate of Films 1-4 was conducted, with measurements taken at 23° C. and 0% relative humidity. Three measurements were taken for each film, in accordance with ASTM D-3985, with the results averaged. Table 5 and FIG. 8 set forth the results of the oxygen transmission rate testing.

TABLE 5
Oxygen Transmission Rate of Films 1-4, 6-12

| | Oxygen Transmission Rate |
|---|---|
| Film | (cc/m² · day · atm) |
| 1 | 30 |
| 2 | 10 |
| 3 | 16 |
| 4 | 10 |
| 6 | 10 |
| 7 | 10 |
| 8 | 10 |
| 9 | 10 |
| 10 | 10 |
| 11 | 10 |
| 12 | 10 |

### Example 6

### Permeation of DMDS Through Films 17-20

Strips measuring 2½″×3½″ were cut from Films 17-20. Each film strip was then folded lengthwise and sealed on two sides to form a 1″ wide pouch. A seal was then formed at the top of each pouch, leaving a ¼″ opening. 1 μL of dimethyl disulfide (DMDS) was added to each pouch through the ¼″ opening using a 10 μL syringe. The dimensions of the finished pouch were 1″×2″. Each pouch was placed into a 22 mL headshape vial. The vial was flushed with nitrogen for 60 seconds to remove any excess DMDS on the outside of the pouch. The vial was then immediately sealed with Teflon-coated aluminum caps.

The concentration of DMDS in the films over time was measured using a fully automated chromatography/mass spectrometry (GC/MS) setup (Thermo Electron Trace GC 2000 with Polaris Q Ion Trap, available from ThermoScientific, Waltham, Mass., United States of America). A DB-624 column, 60M×0.32 mm, 1.8 μm film thickness (available from Agilent Technologies, Santa Clara, Calif., United States of America) was used. An oven temperature profile of 170° C. for 2 minutes, 60° C./minute to 230° C., 2 minute hold was used. A run time of 5 minutes was used. A helium carrier of 1 mL/minute was utilized. A CombiPAL Autosampler (available from CTC Analytics, Zwingen, Switzerland) was used at an incubation temperature of 34° C. for 30 seconds and an injection volume of 250 μL. MS conditions include a full scan 35-150 amu with source temperature of 250° C. The retention time for the DMDS peak was at 4.7 minutes.

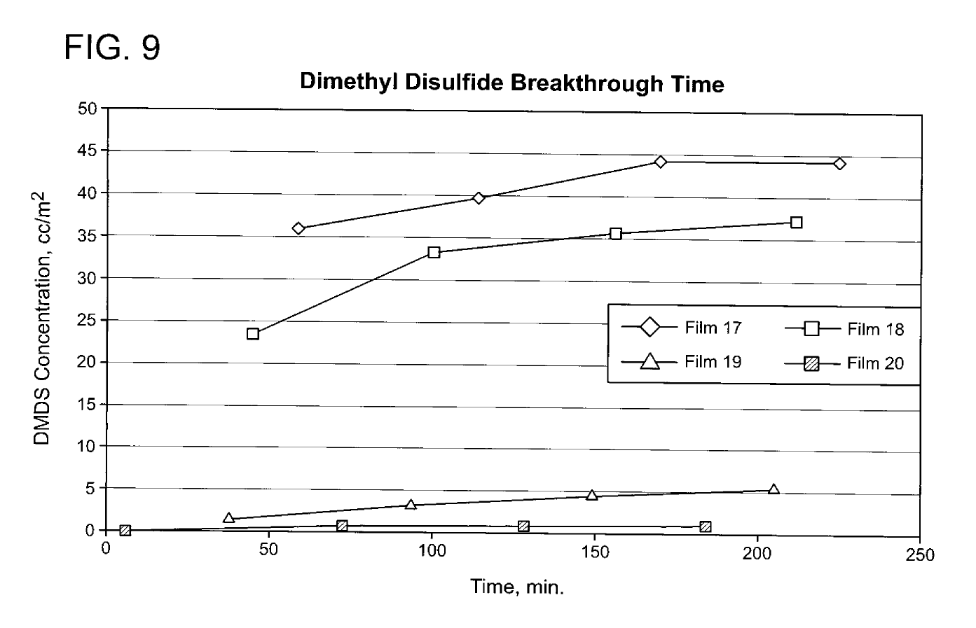

The DMDS concentration over time is given below in Table 6 and represented graphically in FIG. 9.

TABLE 6
DMDS Concentration of Films 17-20

Films

| Time | Film 17 | Film 18 | Film 19 | Film 20 |
|---|---|---|---|---|
| 0 | 0 | 0 | 0 | 0 |
| 6 | — | — | — | 0 |
| 38 | — | — | 1.7 | — |
| 45 | — | 23.4 | — | — |
| 59 | 35.92 | — | — | — |
| 73 | — | — | — | 0.749 |
| 94 | — | — | 3.42 | — |
| 101 | — | 33.16 | — | — |
| 114 | 39.58 | — | — | — |
| 128 | — | — | — | 0.903 |
| 149 | — | — | 4.7 | — |
| 156 | — | 35.36 | — | — |
| 170 | 44.13 | — | — | — |
| 184 | — | — | — | 1.03 |
| 205 | — | — | 5.56 | — |
| 212 | — | 37.19 | — | — |
| 225 | 44.26 | — | — | — |

It can be observed from Table 6 that odor (DMDS) breakthrough as measured in cc/m² was lowest for films 20 and 19, followed by Film 18 and then Film 17.

### Example 7

### Prophetic

### New Barrier Blends

The barrier blends listed below in Table 7 can be added as the odor absorbing barrier layer of any of the films listed herein above, i.e., Films 1-20. The amounts of components V, W, X, and G are given in parts per hundred.

TABLE 7
Barrier Layer Components

Component

| Blend | V | W | X | G |
|---|---|---|---|---|
| 1 | 100 | 2.0 | 0.6 | 0.6 |
| 2 | 100 | 2.0 | 1.0 | 0.6 |
| 3 | 100 | 2.0 | 0.6 | 1.0 |
| 4 | 100 | 2.0 | 1.0 | 1.0 |

What is claimed is:

1. A polymeric film comprising an odor absorbent layer, said odor absorbent layer comprising polyvinylidene chloride polymer and
a. magnesium oxide;
b. magnesium hydroxide;
c. magnesium salt;
d. zeolite; or
e. combinations thereof.

2. The film of claim 1, further comprising an oxygen barrier layer.

3. The film of claim 1, wherein said film comprises a heat sealable layer.

4. The film of claim 1, wherein said zeolite comprises between about 0.1% and 10% by weight of the odor absorbent layer.

5. The film of claim 1, wherein said magnesium oxide comprises between about 0.1% and 10% by weight of the odor absorbent layer.

6. An ostomy pouch comprising a polymeric film comprising an odor absorbent layer, said odor absorbent layer comprising polyvinylidene chloride polymer and
a. magnesium oxide
b. magnesium hydroxide;
c. magnesium salt;
d. zeolite; or
e. combinations thereof.

7. The pouch of claim 6, further comprising an oxygen barrier layer.

8. The pouch of claim 6, wherein said film comprises a heat sealable layer.

9. The pouch of claim 6, wherein said zeolite comprises between about 0.1% and 10% by weight of the odor absorbent layer.

10. The pouch of claim 6, wherein said magnesium oxide comprises between about 0.01% and 10% by weight of the odor absorbent layer.

11. A method of making an ostomy pouch, the method comprising:
a. forming a pouch comprising a first wall and a second wall joined together to define a closed compartment having an interior;
b. providing an aperture formed in one of the first or second walls of the pouch and in communication with the pouch interior;
wherein said first and second walls comprise a polymeric film comprising an odor absorbent layer, said odor absorbent layer comprising polyvinylidene chloride polymer and
a. magnesium oxide;
b. magnesium salt;
c. magnesium hydroxide;
d. zeolite; or
e. combinations thereof.

12. The method of claim 11, further comprising an oxygen barrier layer.

13. The method of claim 11, wherein said film comprises a heat sealable layer.

14. The method of claim 11, wherein said zeolite comprises between about 0.1% and 10% by weight of the odor absorbent layer.

15. The method of claim 11, wherein said magnesium oxide comprises between about 0.1% and 10% by weight of the odor absorbent layer.
